(19) United States
(12) Patent Application Publication
(10) Pub. No.: US 2026/0066128 A1
(43) Pub. Date: Mar. 5, 2026

(54) ESTIMATION METHOD FOR JOINT CAUSAL EFFECTS OF MULTIPLE EXPOSURES BASED ON HIGH-DIMENSIONAL INDEPENDENT VARIABLES

(71) Applicant: Shanxi Medical University, Taiyuan (CN)

(72) Inventors: Qian Gao, Taiyuan (CN); Tong Wang, Taiyuan (CN); Guiming Zhu, Taiyuan (CN)

(21) Appl. No.: 18/817,542

(22) Filed: Aug. 28, 2024

(51) Int. Cl. G16H 50/30; G06F 17/11

(52) U.S. Cl. CPC G16H 50/30; G06F 17/11

(57) ABSTRACT

Disclosed is an estimation method for joint causal effects of multiple exposures based on high-dimensional independent variables, including the following steps: reducing a dimension by using a modified adaptive least absolute shrinkage and selection operator (LASSO); calculating balance weights by using a nonparametric multiple treatments covariate balancing generalized propensity score (npmtCBGPS) method, and determining an optimal value of a tuning parameter by taking a minimum multiple treatment dual-weighted coefficient (mtDWC) as a criterion; and estimating joint causal effects of multiple continuous exposure factors on an outcome variable by using an inverse probability weighting (IPW) method. According to the present invention, in a framework of a GOAL method, a multiple treatments GOAL (mtGOAL) method by combining the npmtCBGPS method with the adaptive LASSO, and a method capable of estimating joint causal effects of multiple continuous exposure factors on an outcome variable in the presence of high-dimensional covariates are proposed.

### TECHNICAL FIELD

The present disclosure relates to the technical field of biotechnology and data analysis, and in particular to an estimation method for joint causal effects of multiple exposures based on high-dimensional independent variables.

### BACKGROUND

In medical research, it is of great guiding significance to identify the causality between treatment or exposure factors and a health outcome and estimate the size of the effect in determining the etiology and exploring the mechanism of disease intervention. In real life, people are usually exposed to multiple potentially dangerous environmental factors at the same time, and these factors work together to affect human health. As nutritional studies suggest, the excessive intake of vitamin A attenuates the association of vitamin D with the risk of death from cancer and cardiovascular disease (Cheng et al., 2012, Cancer Causes Control; Schmutz et al. 2016, Eur J Nutr). As environmental epidemiological studies suggest, the joint exposure to PM2.5, O₃ and NO₂ has a strong correlation with all-cause mortality (Li et al. 2022, Chemosphere). Therefore, simultaneous assessment of the joint causal effects of multiple exposure factors on the health outcome helps to more accurately identify risk factors and ultimately develop public health interventions that meet real-world needs.

The main challenge in estimating causal effects based on observational studies is confounding variables that are associated with exposures and health outcomes, leading to a biased estimate. The generalized propensity score (GPS) method can control the measured confounding factors and estimate a dose-response function (DRF) between a continuous exposure factor and a health outcome. GPS is defined as the conditional probability density of exposure T at a particular value given a pre-exposure covariate X. To obtain the unbiased estimation of causal effects, it is firstly ensured that the correct specification of the GPS model and the conditional probability density function. To relax this condition, the balance-based GPS methods are developed. The method can directly estimate the balance weight by optimizing the balance performance of potential confounding variables across different exposure levels, so it can avoid specifying the GPS model and the conditional probability density function, so it is highly robust and popular in recent years. Representative methods include covariate balancing generalized propensity score (CBGPS), nonparametric covariate balancing generalized propensity score (npCBGPS) and entropy balancing for continuous treatments (EBCT). The simulation results show that the root mean square error (RMSE) of the npCBGPS method is relatively minimal in balance-based methods, regardless of the scenarios including model misspecification, violation of positive assumptions and heterogeneity of treatment effects. However, the method can only estimate a DRF between a single continuous exposure and an outcome. To solve this problem, the npCBGPS method is extended to multiple exposures, which is called a nonparametric multiple treatments covariate balancing generalized propensity score (npmtCBGPS) method. However, the method is unable to select variables. A large number of studies have shown that the optimal GPS model is to include confounding variables and prognostic covariates. The inclusion of unnecessary pre-exposure covariates or the loss of important confounding variables in the model may lead to biased estimates or loss of effectiveness. Therefore, it is necessary to introduce a variable selection technique in the construction of the GPS model, especially in the presence of high-dimensional pre-exposure covariates. For this reason, Gao et al. (2021) proposed generalized outcome-adaptive LASSO (GOAL) method, but it was designed for a single continuous exposure. Therefore, in view of the above deficiencies of the prior art, for those skilled in the art, how to effectively estimate the joint causal effects of multiple continuous exposures on a health outcome in the presence of a high-dimensional covariate is an urgent problem to be solved.

### SUMMARY

An object of the present disclosure is to provide an estimation method for joint causal effects of multiple exposures based on high-dimensional independent variables to solve the problems set forth in the background, and to effectively estimate the joint causal effects of multiple continuous exposure factors on a health outcome without bias in the presence of high-dimensional covariates.

In order to achieve the above object, the present disclosure provides the following technical solutions. An estimation method for joint causal effects of multiple exposures based on high-dimensional independent variables includes the specific following steps:
estimating conditional correlations between each covariate Xⱼ and an outcome variable Y based on generalized covariance measure (GCM);
constructing a GPS model, and selecting covariates that need to be balanced or included in the GPS model by using a modified adaptive least absolute shrinkage and selection operator (LASSO) method;
combining conditional correlations to construct an objective function to solve the GPS model, constructing a multiple treatment dual-weighted coefficient (mtDWC) to select an optimal value of a tuning parameter λₙ in the objective function, and completing variable selection for causal inference;
calculating balance weights by an npmtCBGPS method based on covariates selected by an optimal tuning parameter λd; and
obtaining joint causal effects of multiple continuous exposure factors on an outcome variable by constructing an outcome model of the outcome variable Y being regressed to the exposure factors T using an inverse probability weighting (IPW) method based on the balance weight.

Preferably, the estimating conditional correlations between each covariate Xⱼ and an outcome variable Y based on GCM specifically includes:
assuming:

$${X}_{j} = f ( Z ⋃ {X}_{- j} ) + {ε}_{{X}_{j}} , j = 1 , … p , Y = g ( Z ⋃ {X}_{- j} ) + {ε}_{Y} ,$$

where Z=z(T), z(.) is a known function about exposure factors T, T=(T₁, . . . , Tₘ) represents m-dimensional continuous exposure factors, X=(X₁, . . . , Xₚ) represents p-dimensional pre-exposure covariates, Xⱼ represents a j^{th} pre-exposure covariate, and X₋ⱼ represents a set of other pre-exposure covariates except Xⱼ; and ε_{X}_{ⱼ} and ε_{Y} represent residuals of two models; and f(.) and g(.) represent any linear or non-linear functions, assuming that \hat{f}(Z∪X₋ⱼ) is an estimated value of f(Z∪X₋ⱼ) and ĝ(Z∪X₋ⱼ) is an estimated value of g(Z∪X₋ⱼ), Rᵢⱼ representing a product of the residuals of the two models: Rᵢⱼ=(X₁ⱼ−\hat{f}(Zᵢ∪X₁₋ⱼ))(Yᵢ−ĝ(Zᵢ ∪X₁₋ⱼ)) i=1, 2, . . . n, j=1, . . . p, then GCM being defined as:

$$= \frac{\frac{1}{n} {Σ}_{i = 1}^{n} {R}_{i j}}{{( \frac{1}{n} {Σ}_{i = 1}^{n} {R}_{i j}^{2} - {( \frac{1}{n} {Σ}_{i = 1}^{n} {R}_{i j} )}^{2} )}^{1 / 2}} .$$

Preferably, assuming that the constructed GPS model is a multiple multivariate linear model, the GPS model is represented as: Zᵢ=X₁B+∈ᵢ i=1 . . . n,
where Z=z(T), z(.) with a dimension of r is a known function about exposure factors T, B represents a coefficient matrix with a p*r dimension, ∈ᵢ represents a residual, following a multivariate normal distribution ∈ᵢ˜Nₘ(0, M), and M is a covariance matrix.

Preferably, the objective function is:

$$( \hat{B} , \hat{G} ) = arg {min}_{G , B} \{ tr \{ \frac{1}{n} {( Z - XB )}^{′} ( Z - X B ) G \} - log \vert G \vert + {λ}_{n} {\sum }_{j = 1}^{P} {\sum }_{k = 1}^{r} {w}_{j k} \vert {B}_{j k} \vert \} ,$$

where G=M⁻¹ represents an inverse of a residual covariance matrix;

$${w}_{j .} = {( \frac{\vert \vert}{{max}_{j} \vert \vert} )}^{- γ} ( γ > 1 , j = 1 , … p )$$

represents a penalty weight function, a magnitude of which is inversely proportional to conditional correlations; Bⱼₖ represents an element in a j^{th} row and a k^{th} column of a regression coefficient matrix B; and λₙ>0 indicates a tuning parameter.

Preferably, a set of candidate tuning parameters λₙ satisfying conditions of λₙ/\sqrt{n}→0 and λₙn^{γ/2-1}→∞ are set, and a set of candidate covariate sets are selected based on the candidate tuning parameters λₙ.

Preferably, the mtDWC is represented as:

$$mtDWC ( {λ}_{n} ) = {Σ}_{j = 1}^{p} \vert \vert {Σ}_{k = 1}^{r} E ( {\tilde{w}}_{i}^{{λ}_{n}} {Z}_{i k} {X}_{i j} ) ,$$

where

$$E ( {\tilde{w}}_{i}^{{λ}_{n}} {Z}_{i k} {X}_{i j} )$$

is a weighted correlation coefficient between an exposure function and covariates, reflecting the balance of the covariates,

$${\tilde{w}}_{i}^{{λ}_{n}}$$

being a balance weight estimated by the npmtCBGPS method when a value of the tuning parameter is λₙ, X₁ⱼ representing a value of a j^{th} pre-exposure covariate of an i^{th} individual, and Zᵢₖ representing a value of a k^{th} exposure function of the i^{th} individual; and λₙ corresponding to a minimum value of mtDWC being an optimal tuning parameter.

Preferably, let g(Z(T);θ) represent an estimated DRY, and let θ represent unknown causal parameters; and when there is a linear dose-response relationship between the outcome variable Y and the exposure factors T, Z(T)=T,g(Z(T);θ)=Tθ, at which time the outcome model is expressed as:

$$E [ Y ( t ) ] = T θ = {θ}_{0} + {Σ}_{j = 1}^{m} {θ}_{j} {T}_{j} ,$$

where, Y(t) represents a potential outcome, under causal assumptions that there are no unmeasured confounding assumption (Tᵢ ⊥Yᵢ(t)|X₁, i=1, 2, . . . n), positive assumption (f_{T|X}(Tᵢ=t|X₁)>0, i=1, 2, . . . n), consistency assumption (Yᵢ=Yᵢ(t)) and stable unit value assumption, E[Y(t)]=E[\tilde{w}Y], \tilde{w} represents a balance weight estimated by npmtCBGPS under the optimal λₙ; and at this time, a consistent estimated value \hat{θ} of a causal parameter θ is obtained by using a weighted least square method based on the observed data:

$$\hat{θ} = {argmin}_{θ} {Σ}_{i = 1}^{n} {{\tilde{w}}_{i} ( {Y}_{i} - {T}_{i} θ )}^{2} = {argmin}_{θ} {Σ}_{i = 1}^{n} {{\tilde{w}}_{i} ( {Y}_{i} - {θ}_{0} - {Σ}_{j = 1}^{m} {θ}_{j} {T}_{i j} )}^{2} .$$

The present disclosure provides an estimation method for joint causal effects of multiple exposures based on high-dimensional independent variables, with the following technical effects. In a framework of the GOAL method, a multiple treatments GOAL (mtGOAL) method is proposed by combining the npmtCBGPS method with the adaptive LASSO to effectively estimate the joint causal effects of multiple continuous exposure factors on a health outcome in the presence of high-dimensional covariates without bias. The estimation of joint causal effects is mainly based on the characteristics of complex diseases with multiple causes and high dimension of covariates in health care big data. A large number of statistical simulations show that the overall performance of the mtGOAL method is close to that of ideal method; and when the outcome model or GPS model is linear, on the one hand, it can correctly identify confounding and prognostic covariates, and on the other hand, it retains the finite sample properties of npmtCBGPS method.

### BRIEF DESCRIPTION OF THE DRAWINGS

In order to explain the technical solutions of examples in the present disclosure or the prior art more clearly, the drawings needed in the description of the examples or the prior art are briefly introduced below. Obviously, the attached drawings in the following description are only examples of the present disclosure, and other attached drawings can be obtained according to the provided drawings without creative efforts for those of ordinary skill in the art.

FIG. 1 is a method flow diagram of the present disclosure.

FIG. 2 shows proportions of confounding variables (X₁, X₂ and X₃), prognostic covariates (X₄ and X₅), instrumental variables (X₆, X₇ and X₈), and spurious covariates selected by the mtGOAL method when the outcome model and the GPS model are linear (T2dLY1) with different sample sizes (N=200, 500 and 1000; and ρ=20) and covariate correlation structures (ρ=0, ρ=0.2 and ρ=0.5).

FIG. 3 shows proportions of confounding variables (X₁, X₂ and X₃), prognostic covariates (X₄ and X₅), instrumental variables (X₆, X₇ and XA), and spurious covariates selected by the mtGOAL method when the outcome model is specified correctly (linear) but the GPS model is mis-specified (T2dNLY1) with different sample sizes (N=200, 500 and 1000; and ρ=20) and covariate correlation structures (ρ=0, ρ=0.2 and ρ=0.5).

FIG. 4 shows proportions of confounding variables (X₁, X₂ and X₃), prognostic covariates (X₄ and X₅), instrumental variables (X₆, X₇ and X₅), and spurious covariates selected by the mtGOAL method when the outcome model is mis-specified but the GPS model is specified correctly (T2dLNY1) with different sample sizes (N=200, 500 and 1000; and ρ=20) and covariate correlation structures (ρ=0, ρ=0.2 and ρ=0.5).

FIG. 5 shows proportions of confounding variables (X₁, X₂ and X₃), prognostic covariates (X₄ and X₅), instrumental variables (X₆, X₇ and X₅), and spurious covariates selected by the mtGOAL method when the outcome model and the GPS model are mis-specified (T2dNLNY1) with different sample sizes (N=200, 500 and 1000; and ρ=20) and covariate correlation structures (ρ=0, ρ=0.2 and ρ=0.5).

FIG. 6 shows distributions of causal parameters estimates by the npmtCBGPS method (Targ) and the mtGOAL method when the outcome model and the GPS model are linear (T2dLY1) with different sample sizes (N=200, 500 and 1000; and P=20) and covariate correlation structures (ρ=0, ρ=0.2 and ρ=0.5).

FIG. 7 shows distributions of causal parameters estimates by the npmtCBGPS method (Targ) and the mtGOAL method when the outcome model is specified correctly (linear) but the GPS model is mis-specified (T2dNLY1) with different sample sizes (N=200, 500 and 1000; and P=20) and covariate correlation structures (ρ=0, ρ=0.2 and ρ=0.5).

FIG. 8 shows distributions of causal parameters estimates by the npmtCBGPS method (Targ) and the mtGOAL method when the outcome model is mis-specified, but the GPS model is specified correctly (T2dLNY1) with different sample sizes (N=200, 500 and 1000; and P=20) and covariate correlation structures (ρ=0, ρ=0.2 and ρ=0.5).

FIG. 9 shows distributions of causal parameters estimates by the npmtCBGPS method (Targ) and the mtGOAL method when the outcome model and the GPS model are mis-specified (T2dNLNY1) with different sample sizes (N=200, 500 and 1000; and P=20) and covariate correlation structures (ρ=0, ρ=0.2 and ρ=0.5).

FIG. 10 shows a DRF between PFHxS and BMI with other PFASs taken at medians.

FIG. 11 shows a DRF between PFNA and BMI with other PFASs taken at medians.

FIG. 12 shows a DRF between PFOA and BMI with other PFASs taken at medians.

FIG. 13 shows a DRF between PFOS and BMI with other PFASs taken at medians.

FIG. 14 shows a DRF between PFDA and BMI with other PFASs taken at medians.

### DETAILED DESCRIPTION

Technical solutions in examples of the present disclosure will be described clearly and completely in the following with reference to the attached drawings in the examples of the present disclosure. Obviously, all the described examples are only some, rather than all examples of the present disclosure. Based on the examples in the present disclosure, all other examples obtained by those of ordinary skill in the art without creative efforts belong to the scope of protection of the present disclosure.

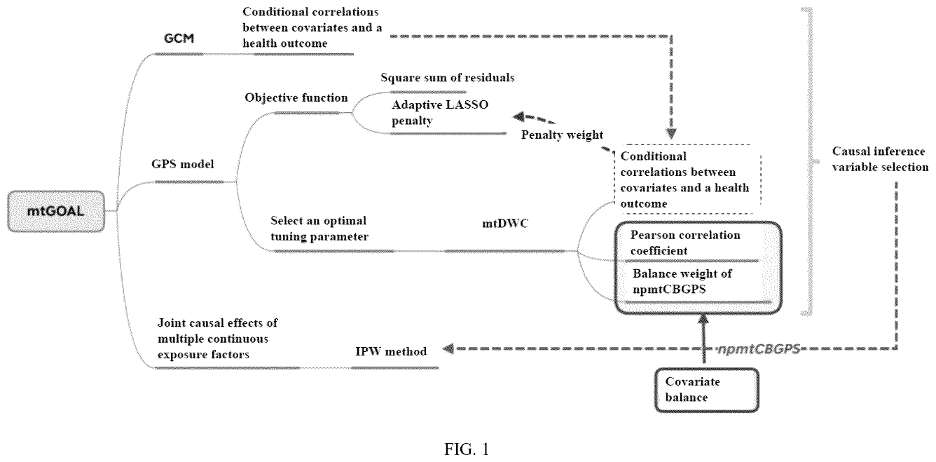

An object of the present disclosure is to provide an estimation method for joint causal effects of multiple exposures based on high-dimensional independent variables, as shown in FIG. 1. A dimension is reduced by using a modified adaptive LASSO; a balance weight is calculated by using the npmtCBGPS method, and an optimal value of tuning parameters is determined by taking a minimum mtDWC as a criterion; and the joint causal effects of multiple continuous exposure factors on outcome variables are estimated by using an IPW method.

The specific technical solutions are as follows.

(1) Conditional correlations between each covariate Xⱼ (j=1, . . . , p) and an outcome variable Y are estimated based on GCM.

It is assumed that:

$${X}_{j} = f ( Z ⋃ {X}_{- j} ) + {ε}_{{X}_{j}} , j = 1 , … p ; \qquad ( 1 )$$

$$Y = g ( Z ⋃ {X}_{- j} ) + {ε}_{Y} ; \qquad ( 2 )$$

where Z=z(T), z(.) is a known function about exposure factors T, T=(T₁, . . . , Tₘ) represents m-dimensional continuous exposure factors, X=(X₁, . . . , Xₚ) represents p-dimensional pre-exposure covariates, Xⱼ represents a j^{th} pre-exposure covariate, and X₋ⱼ represents a set of other pre-exposure covariates except Xⱼ; and ε_{X}_{ⱼ} and ε_{Y} represent residuals of two models; and f(.) and g(.) represent any linear or non-linear functions, assuming that \hat{f}(Z∪X₋ⱼ) is an estimated value of f(Z∪X₋ⱼ) and g(Z∪X₋ⱼ) is an estimated value of g(Z∪X₋ⱼ), Rᵢⱼ representing a product of the residuals of the two models:

$${R}_{i j} = ( {X}_{i j} - \hat{f} ( {Z}_{i} ⋃ {X}_{i - j} ) ) ( {Y}_{i} - \hat{g} ( {Z}_{i} ⋃ {X}_{i - j} ) ) i = 1 , 2 , … n , j = 1 , … p ,$$

then GCM being defined as:

$$= \frac{\frac{1}{n} {Σ}_{i = 1}^{n} {R}_{i j}}{{( \frac{1}{n} {Σ}_{i = 1}^{n} {R}_{i j}^{2} - {( \frac{1}{n} {Σ}_{i = 1}^{n} {R}_{i j} )}^{2} )}^{1 / 2}} . \qquad ( 3 )$$

(2) The causal inference variable selection is achieved based on an adaptive LASSO.

A GPS model (a model with a function Z of exposure factors as a dependent variable) is constructed and a modified adaptive LASSO method is used to select covariates that need to be balanced or included into the GPS model. The present disclosure uses the computed [symbol] described above to construct a penalty weight function. Specifically, the GPS model is assumed to be a multiple multivariate linear model, it is expressed as:

$$\begin{matrix}{Z}_{i} = {X}_{i} B + {ϵ}_{i} & i = 1 … n ;\end{matrix} \qquad ( 4 )$$

where Z=z(T), z(.) with a dimension of r is a known function about exposure factors T, B represents a coefficient matrix with a p*r dimension, ∈ᵢ represents a residual, following a multivariate normal distribution ∈ᵢ˜Nₘ(0,M), and M is a covariance matrix.

The objective function is:

$$( \hat{B} , \hat{G} ) = arg {min}_{G , B} \{ t r \{ \frac{1}{n} {( Z - XB )}^{′} ( Z - X B ) G \} - log \vert G \vert + {λ}_{n} {\sum }_{j = 1}^{P} {\sum }_{k = 1}^{r} {w}_{j k} \vert {B}_{j k} \vert \} \qquad ( 5 )$$

where G=M⁻¹ represents an inverse of a residual covariance matrix;

$${w}_{j .} = {( \frac{\vert \vert}{{max}_{j} \vert \vert} )}^{- γ}$$

$$( γ > 1 , j = 1 , … p )$$

represents a penalty weight function, a magnitude of which is inversely proportional to conditional correlations; Bⱼₖ represents an element in a j^{th} row and a k^{th} column of a regression coefficient matrix B; and λₙ>0 indicates a tuning parameter. To accurately identify confounding variables and prognostic covariates while punishing coefficients of instrumental variables and spurious covariates (including collider variables independent of an outcome and exposure factors) to 0, as in the GOAL method, a set of candidate tuning parameters A, satisfying conditions of λₙ/\sqrt{n}→0 and λₙn^{γ/2-1}→∞ is set, and a set of candidate covariate sets is selected based on the candidate tuning parameters A.

(3) Based on the weak balance condition, an mtDWC is constructed to select an optimal tuning parameter.

Studies have shown that as long as the distribution of confounding variables and prognostic covariates is balanced across different exposure levels, an efficient and consistent effect values can be obtained. Based on this, the present disclosure proposes an mtDWC to select the optimal value of tuning parameters.

$$mtDmc ( {λ}_{n} ) = {\sum }_{j = 1}^{p} \vert \vert {\sum }_{k = 1}^{r} E ( {\tilde{w}}_{i}^{{λ}_{n}} {Z}_{i k} {X}_{ij} ) \qquad ( 6 )$$

where

$$E ( {\tilde{w}}_{i}^{{λ}_{n}} {Z}_{i k} {X}_{i j} )$$

is a weighted correlation coefficient between an exposure function and covariates, reflecting the balance of the covariates,

$${\tilde{w}}_{i}^{{λ}_{n}}$$

being a balance weight estimated by the npmtCBGPS method when a value of the tuning parameter is λₙ, Xᵢⱼ representing a value of a j^{th} pre-exposure covariate of an i^{th} individual, and Zᵢₖ representing a value of a k^{th} exposure function of the i^{th} individual; and λₙ corresponding to a minimum value of the mtDWC being an optimal tuning parameter.

(4) Joint causal effects of multiple continuous exposure factors are estimated by using an IPW method

Using covariates selected by the optimal A, a balance weight is estimated by using an npmtCBGPS method. On this basis, the joint causal effects of multiple continuous exposure factors on an outcome variable are obtained by constructing a weighted linear or non-linear regression model of an outcome variable Y on exposure factors T using the IPW method.

Specifically, taking a linear model as an example, let g(Z(T);θ) represent an estimated DRF, and let θ represent unknown causal parameters; and when there is a linear dose-response relationship between the outcome variable Y and the exposure factors T, Z(T)=T, g(Z(T);θ)=Tθ, at which time the outcome model is expressed as:

$$E [ y ( t ) ] = T θ = {θ}_{0} + {\sum }_{j = 1}^{m} {θ}_{j} {T}_{j} \qquad ( 7 )$$

where, Y(t) represents a potential outcome, under causal assumptions that there are no unmeasured confounding assumption (Tᵢ⊥Yᵢ(t)|Xᵢ, i=1, 2, . . . n), positive assumption (f_{T|X}(Tᵢ=t|Xᵢ)>0, i=1, 2, . . . n), consistency assumption (Yᵢ=Yᵢ(t)) and stable unit value assumption, E[Y(t)]=E[\tilde{w}Y], \tilde{w} represents the balance weights estimated by npmtCBGPS under the optimal λₙ; and at this time, a consistent estimated value \hat{θ} of a causal parameter θ is obtained by using a weighted least square method based on the observed data:

$$\hat{θ} = {arg min}_{θ} {\sum }_{i = 1}^{n} {{\tilde{w}}_{i} ( {y}_{i} - {T}_{i} θ )}^{2} = {arg min}_{θ} {\sum }_{i = 1}^{n} {{\tilde{w}}_{i} ( {Y}_{i} - {θ}_{0} - {\sum }_{j = 1}^{m} {θ}_{j} {T}_{ij} )}^{2} .$$

The present disclosure mainly aims to provide an estimation method for joint causal effects of multiple continuous exposure factors on an outcome variable in the presence of high-dimensional covariates, which is called an mtGOAL method, aiming at the characteristics of multiple etiologies of complex diseases and high dimensionality of covariates in large health care data. A large number of statistical simulations show that the overall performance of the mtGOAL method is close to that of ideal method; and when the outcome model or GPS model is linear, on the one hand, it can correctly identify confounding and prognostic covariates, and on the other hand, it retains the finite sample properties of npmtCBGPS method.

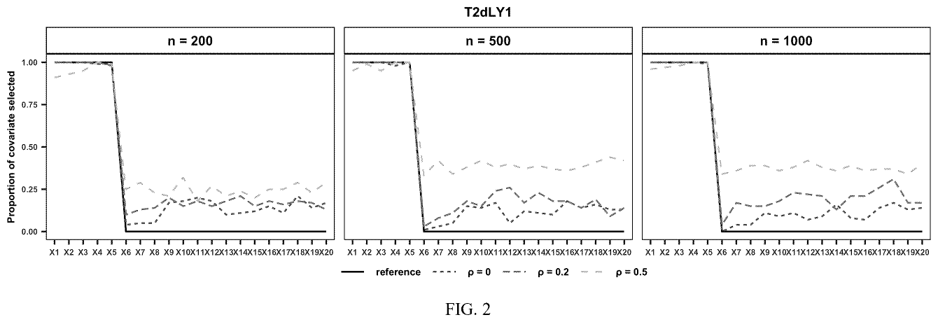

FIGS. 2-5 show performances of the present disclosure in selecting covariates under different simulation scenarios. The exposure factors are 2-dimensional, and have a linear relationship with the health outcome.

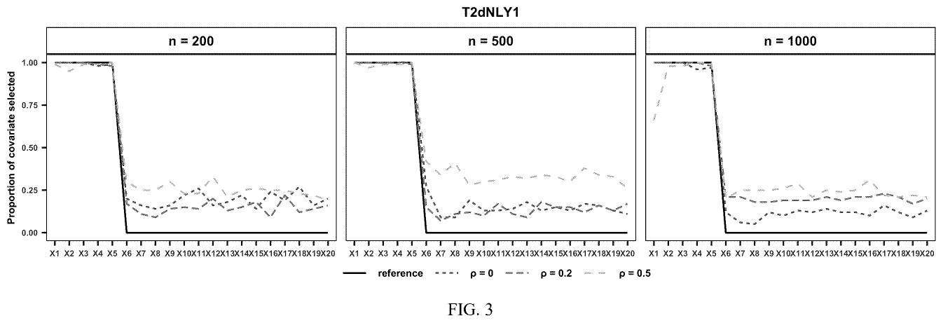

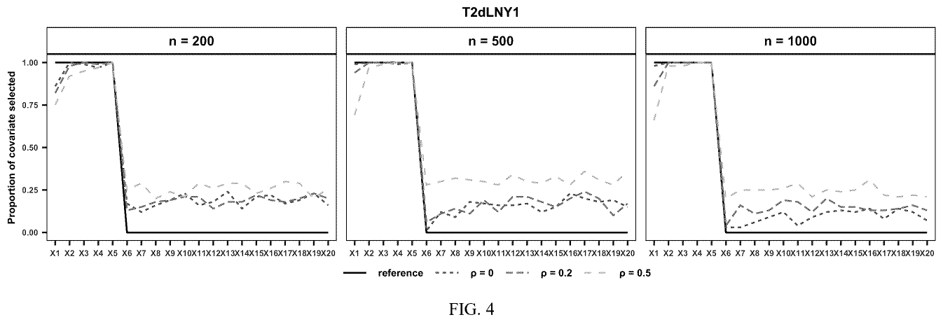

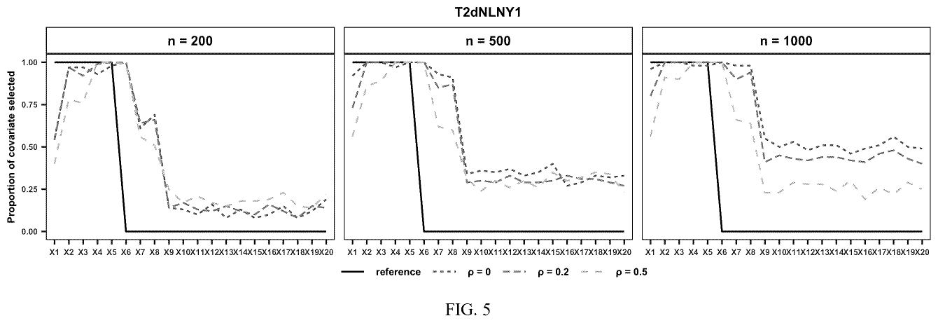

FIG. 2 shows proportions of confounding variables (X₁, X₂ and X₃), prognostic covariates (X₄ and X₅), instrumental variables (X₆, X₇ and X₅), and spurious covariates selected by the mtGOAL method when the outcome model and the GPS model are linear (T2dLY1) with different sample sizes (N=200, 500 and 1000; and ρ=20) and covariate correlation structures (ρ=0, ρ=0.2 and ρ=0.5). FIG. 3 shows proportions of confounding variables (X₁, X₂ and X₃), prognostic covariates (X₄ and X₅), instrumental variables (X₆, X₇ and X₈), and spurious covariates selected by the mtGOAL method when the outcome model is specified correctly (linear) but the GPS model is mis-specified (T2dNLY1) with different sample sizes (N=200, 500 and 1000; and ρ=20) and covariate correlation structures (ρ=0, ρ=0.2 and ρ=0.5). FIG. 4 shows proportions of confounding variables (X₁, X₂ and X₃), prognostic covariates (X₄ and X₅), instrumental variables (X₆, X₇ and X₅), and spurious covariates selected by the mtGOAL method when the outcome model is mis-specified but the GPS model is specified correctly (T2dLNY1) with different sample sizes (N=200, 500 and 1000; and ρ=20) and covariate correlation structures (ρ=0, ρ=0.2 and ρ=0.5). FIG. 5 shows proportions of confounding variables (X₁, X₂ and X₃), prognostic covariates (X₄ and X₅), instrumental variables (X₆, X₇ and X₅), and spurious covariates selected by the mtGOAL method when the outcome model and the GPS model are mis-specified (T2dNLNY1) with different sample sizes (N=200, 500 and 1000; and ρ=20) and covariate correlation structures (ρ=0, ρ=0.2 and ρ=0.5).

The results show that when the outcome model and GPS model are both linear, the mtGOAL method can basically and accurately identify the confounding variables and the prognostic covariates, and the proportions of the instrumental variables and the spurious covariates are selected is close to 0, but increases with the correlation between covariates. When either the outcome model or GPS model is non-linear, the mtGOAL method has the risk of missing confounding variables, and the proportions of the instrumental variables and the spurious covariates are selected is still close to 0, but increases with the correlation between covariates. When both models are non-linear, the mtGOAL method may miss out confounding variables while the proportions of the instrumental variables are selected increases.

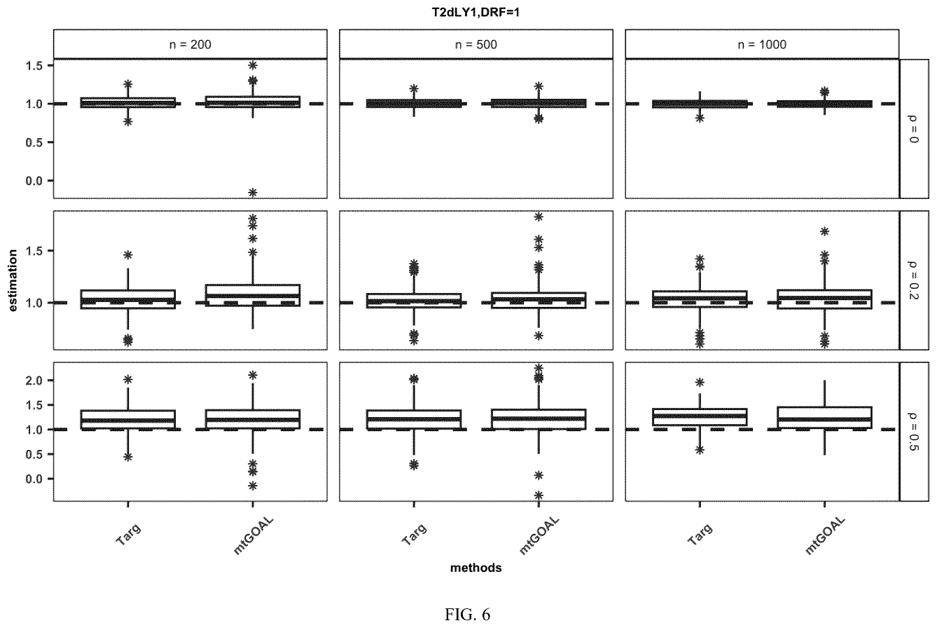

FIGS. 6-9 show distributions of causal parameters estimated values in different simulation scenarios for the mtGOAL method of the present disclosure. Targ is used as a reference to represent the npmtCBGPS method that only balances confounding variables and prognostic covariates. The exposure factors are 2-dimensional and have a linear relationship with a health outcome, and a truth value of the causal parameters is 1.

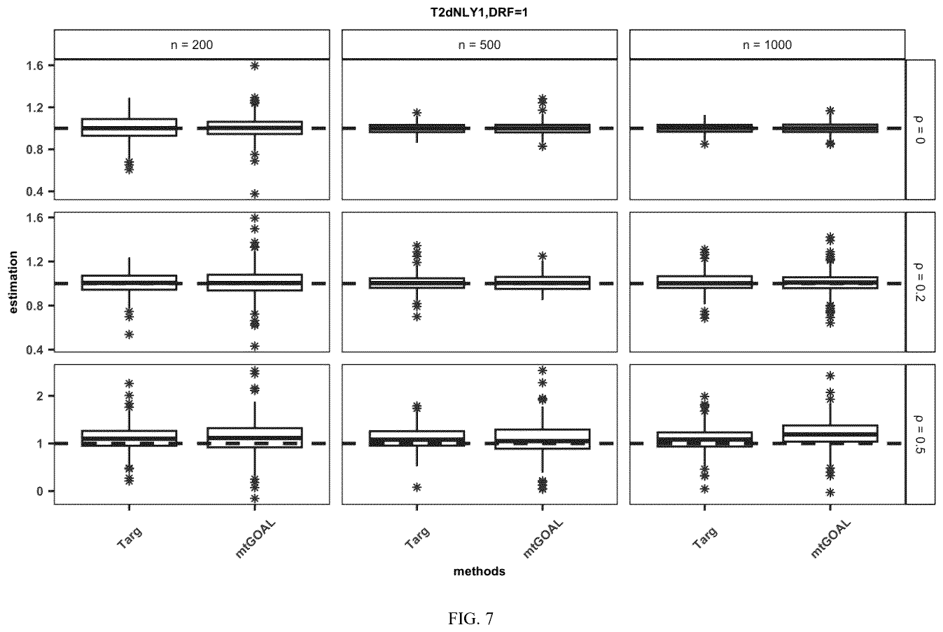

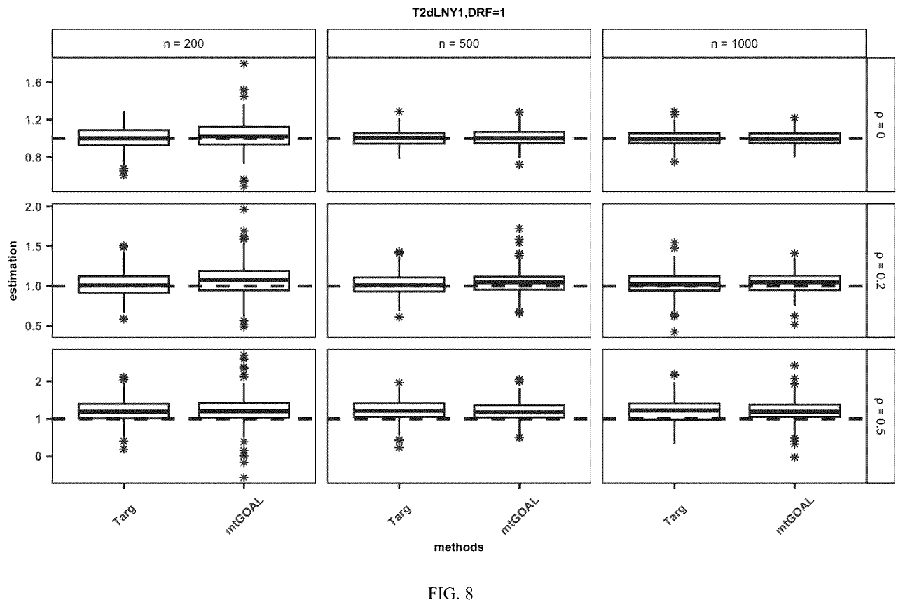

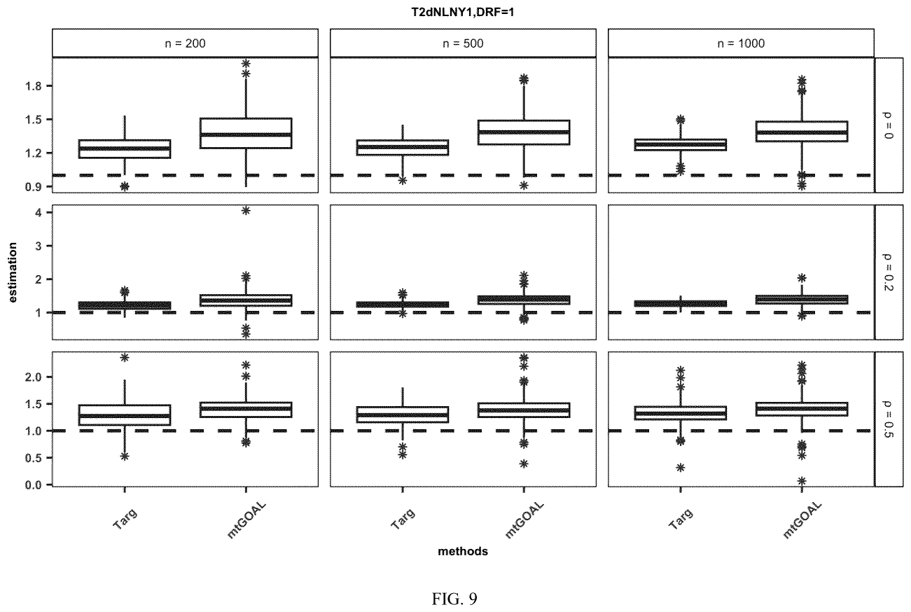

FIG. 6 shows distributions of causal parameters estimated by the npmtCBGPS method (Targ) and the mtGOAL method when the outcome model and the GPS model are linear (T2dLY1) with different sample sizes (N=200, 500 and 1000; and P=20) and covariate correlation structures (ρ=0, ρ=0.2 and ρ=0.5). FIG. 7 shows distributions of causal parameters estimated by the npmtCBGPS method (Targ) and the mtGOAL method when the outcome model is specified correctly (linear) but the GPS model is mis-specified (T2dNLY1) with different sample sizes (N=200, 500 and 1000; and P=20) and covariate correlation structures (ρ=0, ρ=0.2 and ρ=0.5). FIG. 8 shows distributions of causal parameters estimated by the npmtCBGPS method (Targ) and the mtGOAL method when the outcome model is mis-specified, but the GPS model is specified correctly (T2dLNY1) with different sample sizes (N=200, 500 and 1000; and P=20) and covariate correlation structures (ρ=0, ρ=0.2 and ρ=0.5). FIG. 9 shows distributions of causal parameters estimated by the npmtCBGPS method (Targ) and the mtGOAL method when the outcome model and the GPS model are mis-specified (T2dNLNY1) with different sample sizes (N=200, 500 and 1000; and P=20) and covariate correlation structures (ρ=0, ρ=0.2 and ρ=0.5).

The results show that the performance of mtGOAL method is close to Targ. When either the outcome model or the GPS model is linear, the estimated value of the causal parameter of the mtGOAL method is close to the true value, but when both of the two models are non-linear, the bias of the estimated value will become larger, that is, the mtGOAL method shows properties similar to double robustness. The performance of the mtGOAL method is influenced by the correlation structure of covariates and sample sizes: when a sample size is small, the estimation accuracy and precision of the mtGOAL method are slightly worse than those of the Targ method. As the sample size increases, the two methods tend to be consistent, and the estimation accuracy and precision improve. However, as the correlation between covariates increases, the estimation bias increases, and the estimation precision decreases.

Further, an application of the mtGOAL method is exemplified by exploring the dose-response relationship between perfluoroalkyl and polyfluoroalkyl substances (PFASs) and the body mass index (BMI). The data comes from the National Nutrition Health and Examination Survey (NHANES) in 2015-2018.

PFASs with a detection rate greater than 80% are retained and ultimately included perfluorooctanoic acid (PFOA), perfluorooctane sulfonate (PFOS), perfluorononanoic acid (PFNA), perfluorodecanoic acid (PFDA), and perfluorohexane-1-sulphonic acid (PFHxS). A total of 64 potential confounding variables are considered, including demographic characteristics such as age, gender, race and educational level; blood biochemical indicators such as high density lipoprotein and total cholesterol; and environmental exposures such as dimethyl phosphate, diethyl phosphate, mono (carboxyl nonyl) phthalate, and mono (carboxyl octyl) phthalate. The results of linear analysis show that there is a statistically significant linear dose-response relationship between PFNA and BMI; and when PFNA increases by 2.72 ng/ml, BMI increases by 1.66 kg/m² on average. There is no statistically significant linear dose-response relationship between other PFASs and BMI, as shown in Table 1.

TABLE 1

| Variable | DRF | Std. Error | t value | P-value |
|---|---|---|---|---|
| (Intercept) | 30.78 | 1.15 | 26.80 | <0.0001 |
| InPFDA | −0.65 | 0.49 | −1.33 | 0.18 |
| InPFHxS | 0.19 | 0.50 | 0.38 | 0.70 |
| InPFNA | 1.66 | 0.56 | 2.93 | 0.003 |
| InPFOA | −0.54 | 0.69 | −0.78 | 0.44 |
| InPFOS | −0.63 | 0.48 | −1.31 | 0.19 |

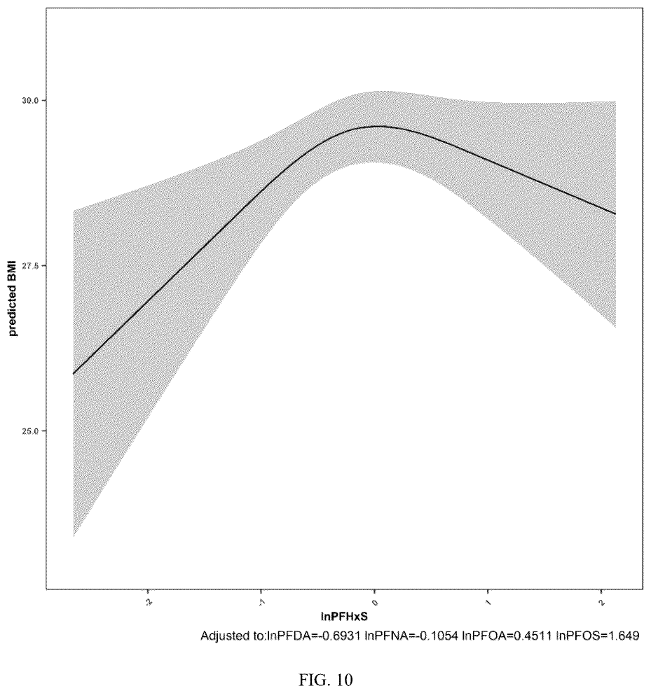

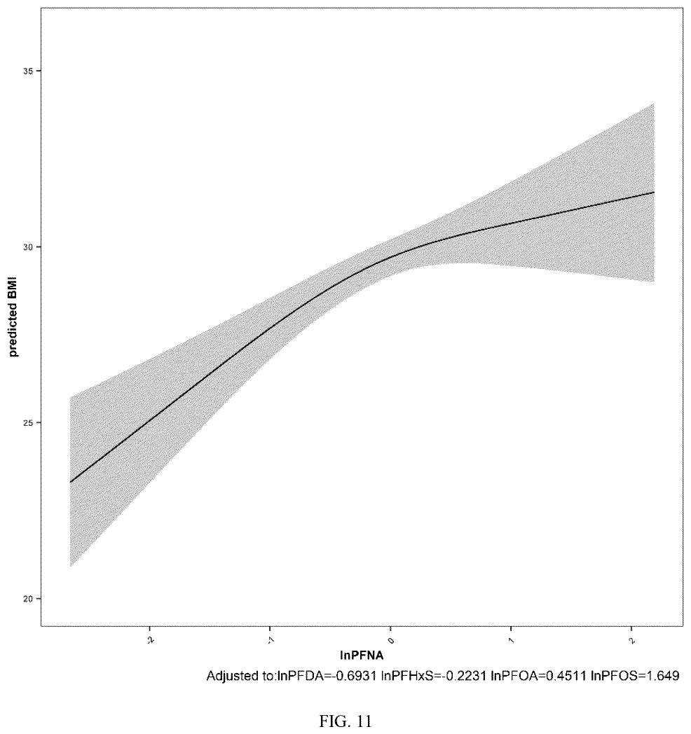

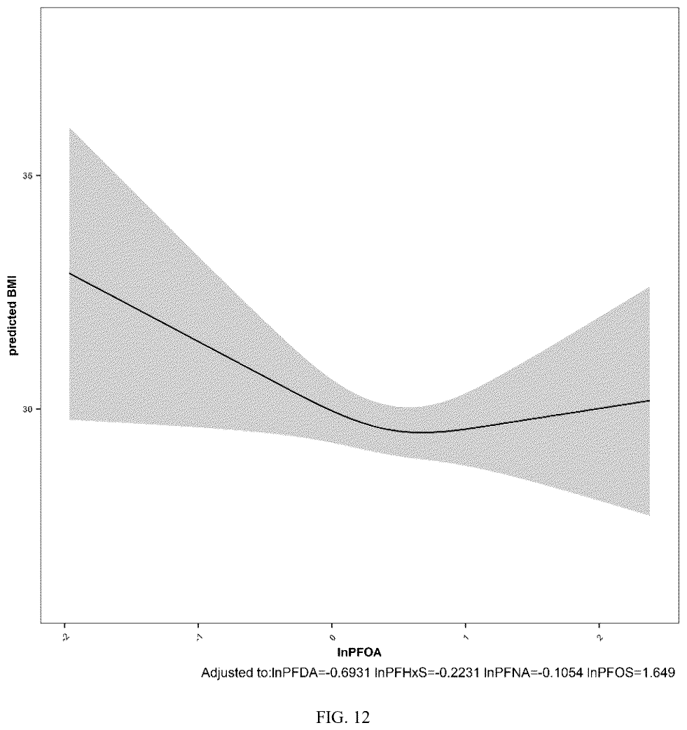

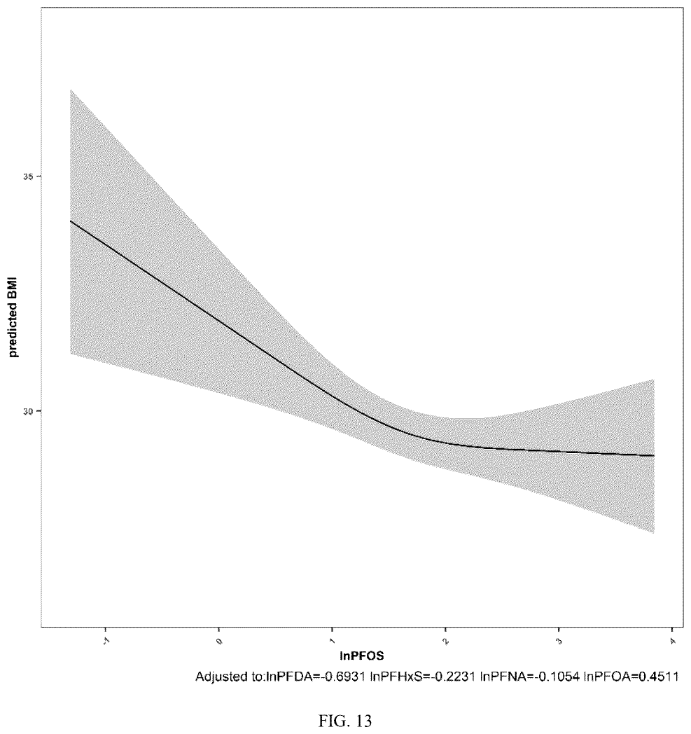

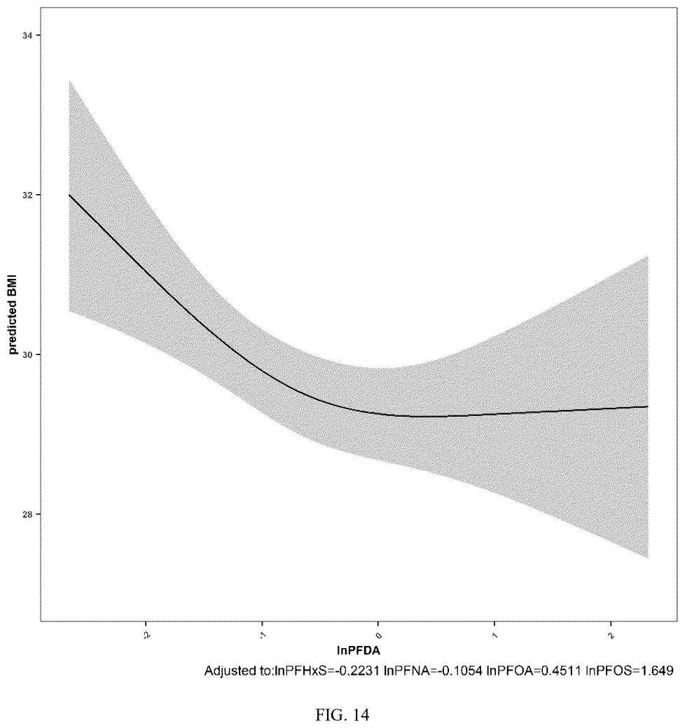

The results of non-linear analysis are shown in FIGS. 10-14. FIG. 10 shows a DRF between PFHxS and BMI with other PFASs taken at medians. FIG. 11 shows a DRF between PFNA and BMI with other PFASs taken at medians. FIG. 12 shows a DRF between PFOA and BMI with other PFASs taken at medians. FIG. 13 shows a DRF between PFOS and BMI with other PFASs taken at medians. FIG. 14 shows a DRF between PFDA and BMI with other PFASs taken at medians.

The results show that BMI increases with the increase of PFNA, PFHxS shows an inverted U-shaped trend, while PFDA, PFOA and PFOS show a U-shaped trend.

Herein, specific examples are used to explain the principle and implementation of the present disclosure, and the description of the above examples is only used to help understand the method and its core idea of the present disclosure. At the same time, for those of ordinary skill in the art, many changes can be made in the specific implementations and application scopes according to the idea of the present disclosure. In view of the above, this description is not to be construed as limiting the present disclosure.

1. An estimation method for joint causal effects of multiple exposures based on high-dimensional independent variables, comprising the specific following steps:
estimating conditional correlations between each covariate Xⱼ and an outcome variable Y based on generalized covariance measure (GCM);
constructing a generalized propensity score (GPS) model, and selecting covariates that need to be balanced or included in the GPS model by using a modified adaptive least absolute shrinkage and selection operator (LASSO) method;
combining conditional correlations to construct an objective function to solve the GPS model, constructing a multiple treatment dual-weighted coefficient (mtDWC) to select an optimal value of a tuning parameter λₙ in the objective function, and completing variable selection for causal inference;
calculating balance weights by a nonparametric multiple treatments covariate balancing generalized propensity score (npmtCBGPS) method based on covariates selected by the optimal tuning parameter λₘ; and
obtaining joint causal effects of multiple continuous exposure factors on an outcome variable by constructing an outcome model of the outcome variable Y being regressed to the exposure factors T using an inverse probability weighting (IPW) method based on the balance weight.

2. The estimation method for joint causal effects of multiple exposures based on high-dimensional independent variables according to claim 1, wherein the estimating conditional correlations between each covariate Xⱼ and an outcome variable Y based on GCM specifically comprises:
assuming:
$${X}_{j} = f ( Z ⋃ {X}_{- j} ) + {ε}_{{X}_{j}} , j = 1 , … p , Y = g ( Z ⋃ {X}_{- j} ) + {ε}_{Y} ,$$
where Z=z(T), z(.) is a known function about exposure factors T, T=(T₁, . . . , Tₘ) represents m-dimensional continuous exposure factors, X=(X₁, . . . , Xₚ) represents p-dimensional pre-exposure covariates, X₁ represents a j^{th} pre-exposure covariate, and X₋ⱼ represents a set of other pre-exposure covariates except Xⱼ; and ε_{X}_{ⱼ} and ε_{γ} represent residuals of two models; and f(.) and g(.) represent any linear or non-linear functions, assuming that \hat{f}(Z∪X₋ⱼ) is an estimated value of f(Z∪X₋ⱼ) and g(Z∪X₋ⱼ) is an estimated value of g(Z∪X₋ⱼ), R representing a product of the residuals of the two models: Rᵢⱼ=(X₁ⱼ−\hat{f}(Zᵢ∪X₁₋ⱼ))(Yᵢ−ĝ(Zᵢ∪X₁₋ⱼ)) i=1, 2, . . . n, j=1, . . . p, then GCM being defined as:
$$= \frac{\frac{1}{n} {\sum }_{i = 1}^{n} {R}_{i j}}{{( \frac{1}{n} {\sum }_{i = 1}^{n} {R}_{i j}^{2} - {( \frac{1}{n} {\sum }_{i = 1}^{n} {R}_{i j} )}^{2} )}^{1 / 2}} .$$

3. The estimation method for joint causal effects of multiple exposures based on high-dimensional independent variables according to claim 2, wherein assuming that the constructed GPS model is a multiple multivariate linear model, the GPS model is represented as: Zᵢ=X₁B+∈ᵢ i=1 . . . n,
where Z=z(T), z(.) with a dimension of r is a known function about exposure factors T, B represents a coefficient matrix with a p*r dimension, ∈ᵢ represents a residual, following a multivariate normal distribution ∈ᵢ˜Nₘ(0,M), and Mis a covariance matrix.

4. The estimation method for joint causal effects of multiple exposures based on high-dimensional independent variables according to claim 3, wherein the objective function is:
$$( \hat{B} , \hat{G} ) = arg {min}_{G , B} \{ t r \{ \frac{1}{n} {( Z - XB )}^{′} ( Z - X B ) G \} - log \vert G \vert + {λ}_{n} {\sum }_{j = 1}^{P} {\sum }_{k = 1}^{r} {w}_{j k} \vert {B}_{j k} \vert \} ,$$
where G=M⁻¹ represents an inverse of a residual covariance matrix;
$${w}_{j .} = {( \frac{\vert \vert}{{max}_{j} \vert \vert} )}^{- γ}$$
$$( γ > 1 , j = 1 , … p )$$
represents a penalty weight function, a magnitude of which is inversely proportional to conditional correlations; Bⱼₖ represents an element in a j^{th} row and a k^{th} column of a regression coefficient matrix B; and λₙ>0 indicates a tuning parameter.

5. The estimation method for joint causal effects of multiple exposures based on high-dimensional independent variables according to claim 1, wherein a set of candidate tuning parameters λₙ satisfying conditions of λₙ/\sqrt{n}→0 and λₙn^{γ/2-1}→∞ are set, and a set of candidate covariate sets are selected based on the candidate tuning parameters λₙ.

6. The estimation method for joint causal effects of multiple exposures based on high-dimensional independent variables according to claim 4, wherein the mtDWC is represented as:
$$mtDWC ( {λ}_{n} ) = {\sum }_{j = 1}^{p} \vert \vert {\sum }_{k = 1}^{r} E ( {\tilde{w}}_{i}^{{λ}_{n}} {Z}_{i k} {X}_{i j} ) ,$$
where
$$E ( {\tilde{w}}_{i}^{{λ}_{n}} {Z}_{i k} {X}_{i j} )$$
is a weighted correlation coefficient between an exposure function and covariates, reflecting the balance of the covariates,
$${\tilde{w}}_{i}^{{λ}_{n}}$$
being a balance weight estimated by the npmtCBGPS method when a value of the tuning parameter is λₙ, X₁ⱼ representing a value of a j^{th} pre-exposure covariate of an i^{th} individual, and Zᵢₖ representing a value of a k^{th} exposure function of the i^{th} individual; and λₙ corresponding to a minimum value of the mtDWC being the optimal adjustment parameter.

7. The estimation method for joint causal effects of multiple exposures based on high-dimensional independent variables according to claim 1, wherein let g(Z(T);θ) represent an estimated dose-response function (DRY), and let θ represent unknown causal parameters; and when there is a linear dose-response relationship between the outcome variable Y and the exposure factors T, Z(T)=T, g(Z(T);θ)=Tθ, at which time the outcome model is expressed as:
$$E [ y ( t ) ] = T θ = {θ}_{0} + {\sum }_{j = 1}^{m} {θ}_{j} {T}_{j} ,$$
where, Y(t) represents a potential outcome, under causal assumptions that there are no unmeasured confounding assumption (Tᵢ⊥Yᵢ(t)|X₁, i=1, 2, . . . n), positive assumption (f_{T|X}(Tᵢ=t|X₁)>0, i=1, 2, . . . n), consistency assumption (Yᵢ=Yᵢ(t)) and stable unit value assumption, E[Y(t)]=E[\tilde{w}Y], \tilde{w} represents balance weights estimated by npmtCBGPS under the optimal λₙ; and at this time, a consistent estimated value \hat{θ} of a causal parameter θ is obtained by using a weighted least square method based on the observed data:
$$\hat{θ} = {arg min}_{θ} {\sum }_{i = 1}^{n} {{\tilde{w}}_{i} ( {y}_{i} - {T}_{i} θ )}^{2} = {arg min}_{θ} {\sum }_{i = 1}^{n} {{\tilde{w}}_{i} ( {Y}_{i} - {θ}_{0} - {\sum }_{j = 1}^{m} {θ}_{j} {T}_{ij} )}^{2} .$$
